(19) United States
(12) Patent Application Publication
(10) Pub. No.: US 2015/0044159 A1
(43) Pub. Date: Feb. 12, 2015

(54) MALODOR CONTROL COMPOSITION FOR USE IN-HOME COMPOSTING

(71) Applicant: The Procter & Gamble Company, Cincinnati, OH (US)

(72) Inventors: Jennifer Melissa Ross DEVINE, Cincinnati, OH (US); Brent William MASON, West Chester, OH (US)

(21) Appl. No.: 14/522,611

(22) Filed: Oct. 24, 2014

Related U.S. Application Data
(63) Continuation of application No. PCT/US2013/037780, filed on Apr. 23, 2013.
(60) Provisional application No. 61/638,513, filed on Apr. 26, 2012.
(60) Provisional application No. 61/644,554, filed on May 9, 2012.

(51) Int. Cl. C05F 17/00; A61L 9/01; C05F 17/02

(52) U.S. Cl. CPC C05F 17/00; C05F 17/0063; C05F 17/0264; A61L 9/01; A61L 2209/13; A61L 2209/21
USPC 424/76.1; 71/8; 435/290.1

(57) ABSTRACT

The use of a malodor control composition in a home composting appliance helps to eliminate malodor emitting from compostable materials contained in the appliance during the composting process.

### FIELD OF THE INVENTION

The present invention in general is related to in-home composting.

### BACKGROUND OF THE INVENTION

There are in-home composting appliances that are commercially available. The benefits of composting include the reduction of waste in landfills and an economical source of plant food. Malodor emitted as a result of in-home composting is a barrier for broad consumer appeal and adoption. One approach for mitigating malodor is to have an exhaust tube connected from the appliance to a vent venting to outside the home. The disadvantage to the approach is the cost and complexity of installing such a venting system. Another approach is the use of a platinum/cadmium catalyst to reduce malodor. The disadvantage is the cost wherein the retail price of these appliances is offered to at over 1,000 USD. Yet another approach is the use of a carbon filter. Often these filter systems are not completely effective in removing the malodor or require large, noisy, and expensive blower systems to work effectively.

There is a need for a cost effective and simple system to eliminate or reduce malodor emitted from composting, particularly through in-home composting appliances, without covering or masking the malodor.

### SUMMARY OF THE INVENTION

The present invention attempts to solve these needs by the use of malodor control compositions (“MCC”). In one aspect, MCC may be used in a passive emitter or active emitter in the home composting appliance. In another aspect, MCC may be injected into the container containing the compostable materials of the composting appliance. In yet another aspect, MCC is incorporated in an air filter filtering air passing through one or more vents of the composting appliance.

A first aspect of the invention provides a composting appliance. The appliance comprises: at least one container capable of containing compostable materials; a heater configured to heat the container or the compostable materials contained in the container, and a passive or energized malodor control composition dispensing device configured to dispense a malodor control composition.

A second aspect of the invention provides for a method of eliminating odor comprising the steps: providing a container configured to receive compostable materials; containing compostable materials in the container; composting the compostable materials contained in the container; emitting malodor from the container containing the compostable materials; dispensing a malodor control composting in fluid communication the compostable materials contained in the container to eliminate the emitted malodor.

A third aspect of the invention provides for a method of eliminating odor in a home composting appliance comprising the steps; providing a unit dose article comprising a malodor control composition; administering the article into a container of a home composting appliance wherein the container is capable of containing compostable materials.

### DETAILED DESCRIPTION OF THE INVENTION

The present invention relates to a MCC comprising at least one volatile aldehyde and optionally an acid catalyst, low molecular weight polyols, cyclodextrin, buffer agent, solubilizer, antimicrobial compound, aqueous carrier, and combinations thereof, and methods of using MCC in association with composting, preferably in association with an in-home composting appliance.

“Malodor” refers to compounds generally offensive or unpleasant to most people that may be emitted from in-home composting.

“Neutralize” or “neutralization” refers to the ability of a compound or product to reduce or eliminate malodorous compounds. Odor neutralization may be partial, affecting only some of the malodorous compounds in a given context, or affecting only part of a malodorous compound. A malodorous compound may be neutralized by chemical reaction resulting in a new chemical entity, by sequestration, by chelation, by association, or by any other interaction rendering the malodorous compound less malodorous or non-malodorous. Odor neutralization may be distinguished from odor masking or odor blocking by a change in the malodorous compound, as opposed to a change in the ability to perceive the malodor without any corresponding change in the condition of the malodorous compound.

“Composting Appliance” is an appliance that may be used inside a structure (e.g., home or place of business) for the purpose to converting compostable materials (like foods scraps) to compost. The composting appliance typically will mix/provide heat to the compostable material. Composting microbes, composting enzymes, probiotics, and other ingredients may be added to the composting appliance to facilitate the composting process. A non-limiting example of a composting appliance is described in US 2008/0209967 A1.

The MCC of the present invention may be used in a wide variety of applications in the context of in-home composting that neutralize malodors in the vapor and/or liquid phase. In some embodiments, the malodor control composition may be formulated for use in energized vapor phase systems. “Energized” as used herein refers to a system that operates by using an electrical energy source to emit a targeted active. For such systems, the VP of the volatile aldehydes may be about 0.001 torr to about 20 torr, alternatively about 0.01 torr to about 10 torr, measured at 25° C. One example of an energized vapor phase system is a liquid electric air freshening device. Non-limiting examples of an energized system include a wick system (preferably heating the wick or composition per U.S. Pat. No. 7,223,361), vibration (e.g., ultrasonic or piezoelectric per US 2011/0266359 A1), or combinations thereof.

In some embodiments, the malodor control composition may be formulated for use in non-energized vapor phase systems. “Non-energized” as used herein refers to a system that emits a MCC passively or without the need for an electrical energy source. Sprayers or injectors are considered non-energized systems. Of course, spraying or injected may be automated (i.e., motorized) and yet still be considered “non-energized” for purposes of the present invention. For such non-energized systems, the VP of the volatile aldehydes may be about 0.01 torr to about 20 torr, alternatively about 0.05 torr to about 10 torr, measured at 25° C.

In other embodiments, the malodor control composition may be formulated for use in a liquid phase system. For such systems, the VP may be about 0 torr to about 20 torr, alternatively about 0.0001 torr to about 10 torr, measured at 25° C. Non-limiting examples of a liquid phase system are direct application into the compostable material contained in one or more of the containers of the composting appliance.

The MCC may be contained in a vial for dispensing in the composting appliance. The term “vial” is broadly defined to include container that are generally suitable to contain perfume composition. A non-limiting example of a vial includes a scented oil refills for FEBREZE NOTICEABLES (P&G). In one embodiment, the vial contains from about 5 ml to about 250 ml, alternatively from 25 ml to about 125 ml, alternatively from about 50 ml to about 150 ml, alternatively combinations thereof. The vial may be plastic or glass or combination thereof. The vial may be a consumable, i.e., replaceable by the user as it becomes depleted.

The MCC may also be formulated for use in substrates such as plastics, wovens, or non-wovens (e.g., cellulose fibers for paper products). Such an application may be useful for air filters for the composting appliance. The air filter may be functionally attached to a vent of the composting appliance. The filter may be configured to filter air venting from a vent of the appliance. In another embodiment, the MCC may be integrated as a component of an activated carbon air filter.

The malodor control composition includes a mixture of volatile aldehydes and is designed to deliver genuine malodor neutralization and not function merely by covering up or masking odors. A genuine malodor neutralization provides a sensory and analytically measurable (e.g. gas chromatograph) malodor reduction. Thus, if the malodor control composition delivers genuine malodor neutralization, the composition will reduce malodors in the vapor and/or liquid phase.

The malodor control composition includes a mixture of volatile aldehydes that neutralize malodors in vapor and/or liquid phase via chemical reactions. Such volatile aldehydes are also called reactive aldehydes (RA). Volatile aldehydes may react with amine-based odors, following the path of Schiff-base formation. Volatiles aldehydes may also react with sulfur-based odors, forming thiol acetals, hemi thiolacetals, and thiol esters in vapor and/or liquid phase. It may be desirable for these vapor and/or liquid phase volatile aldehydes to have virtually no negative impact on the desired perfume character of a product. Aldehydes that are partially volatile may be considered a volatile aldehyde as used herein.

Suitable volatile aldehydes may have a vapor pressure (VP) in the range of about 0.0001 ton to 100 torr, alternatively about 0.0001 torr to about 10 torr, alternatively about 0.001 torr to about 50 torr, alternatively about 0.001 torr to about 20 torr, alternatively about 0.001 torr to about 0.100 torr, alternatively about 0.001 torr to 0.06 torr, alternatively about 0.001 torr to 0.03 ton, alternatively about 0.005 torr to about 20 torr, alternatively about 0.01 torr to about 20 torr, alternatively about 0.01 torr to about 15 torr, alternatively about 0.01 torr to about 10 torr, alternatively about 0.05 torr to about 10 torr, measured at 25° C.

The perfume compositions can include ingredients that are suitably used for home composting. The perfume ingredients are not limited but can be selected based on their Kovat's Index (“KI”) (as determined on 5% phenyl-methylpolysiloxane as non-polar silicone stationary phase). The KI places the volatility attributes of an analyte (e.g. component of a volatile composition) on a gas chromatography column in relation to the volatility characteristics of an n-alkane (normal alkane) series on that column. A typical gas chromatograph (“GC”) column is a DB-5 column available from Agilent Technologies of Palo Alto, California. By this definition, the KI of a normal alkane is set to 100n, where n is the number of carbon atoms in the n-alkane. The KI of an analyte, x, eluting at time t′, between two n-alkanes with number of carbon atoms “n” and “N” having corrected retention times t′ₙ and t′_{N} respectively, will then be calculated as:

$$KI = 100 ( n + \frac{log {t}_{x}^{′} - log {t}_{n}^{′}}{log {t}_{N}^{′} - log {t}_{n}^{′}} )$$

On a non-polar to slightly polar GC stationary phase, KI of analytes are correlated with their relative volatility. For example, analytes with smaller KIs tend to be more volatile than those with larger KIs. Ranking analytes with their corresponding KI values gives a good comparison of analyte evaporation rates in liquid-gas partitioning systems. The volatile composition according to the present invention can have at least one ingredient with a KI value of about 600 to about 1800, or about 800 to about 1700, or about 900 to about 1600. The volatile composition can comprise about 50% to about 100%, or about 70% to about 100%, or about 80% to about 100% of one or more ingredients having these KI values.

Rather than, or in addition to Kovat's Index, the perfume ingredients can be selected based on their boiling point (or “B.P.”) and their octanol/water partition coefficient (or “P”). The boiling point referred to herein is measured under normal standard pressure of 760 mm Hg. The boiling points of many perfume ingredients, at standard 760 mm Hg can be found in “Perfume and Flavor Chemicals (Aroma Chemicals),” written and published by Steffen Arctander, 1969. In one embodiment, the perfume comprises from about 50% to about 100% by weight of at least one perfume ingredient, alternatively two, three, four, five, or more perfume ingredients, which has (have) a Kovat's Index from about 600 to about 1800.

The octanol/water partition coefficient of a volatile aldehyde is the ratio between its equilibrium concentrations in octanol and in water. The partition coefficients of the volatile aldehydes used in the malodor control composition may be more conveniently given in the form of their logarithm to the base 10, logP. The logP values of many volatile aldehydes have been reported. See, e.g., the Pomona92 database, available from Daylight Chemical Information Systems, Inc. (Daylight CIS), Irvine, Calif. (latest version or edition). However, the logP values are most conveniently calculated by the “CLOGP” program, also available from Daylight CIS. This program also lists experimental logP values when they are available in the Pomona92 database. The “calculated logP” (ClogP) is determined by the fragment approach of Hansch and Leo (cf., A. Leo, in Comprehensive Medicinal Chemistry, Vol. 4, C. Hansch, P. G. Sammens, J. B. Taylor and C. A. Ramsden, Eds., p. 295, Pergamon Press, 1990). The fragment approach is based on the chemical structure of each volatile aldehyde, and takes into account the numbers and types of atoms, the atom connectivity, and chemical bonding. The ClogP values, which are the most reliable and widely used estimates for this physicochemical property, are preferably used instead of the experimental logP values in the selection of volatile aldehydes for the malodor control composition.

The ClogP values may be defined by four groups and the volatile aldehydes may be selected from one or more of these groups. The first group comprises volatile aldehydes that have a B.P. of about 250° C. or less and ClogP of about 3 or less. The second group comprises volatile aldehydes that have a B.P. of 250° C. or less and ClogP of 3.0 or more. The third group comprises volatile aldehydes that have a B.P. of 250° C. or more and ClogP of 3.0 or less. The fourth group comprises volatile aldehydes that have a B.P. of 250° C. or more and ClogP of 3.0 or more. The malodor control composition may comprise any combination of volatile aldehydes from one or more of the ClogP groups.

In some embodiments, the malodor control composition of the present invention may comprise, by total weight of the malodor control composition, from about 0% to about 30% of volatile aldehydes from group 1, alternatively about 25%; and/or about 0% to about 10% of volatile aldehydes from group 2, alternatively about 10%; and/or from about 10% to about 30% of volatile aldehydes from group 3, alternatively about 30%; and/or from about 35% to about 60% of volatile aldehydes from group 4, alternatively about 35%. Exemplary volatile aldehydes which may be used in a malodor control composition include, but are not limited to, Adoxal (2,6,10-Trimethyl-9-undecenal), Bourgeonal (4-t-butylbenzenepropionaldehyde), Lilestralis 33 (2-methyl-4-t-butylphenyl)propanal), Cinnamic aldehyde, cinnamaldehyde (phenyl propenal, 3-phenyl-2-propenal), Citral, Geranial, Neral (dimethyloctadienal, 3,7-dimethyl-2,6-octadien-1-al), Cyclal C (2,4-dimethyl-3-cyclohexen-1-carbaldehyde), Florhydral (3-(3-Isopropyl-phenyl)-butyraldehyde), Citronellal (3,7-dimethyl 6-octenal), Cymal, cyclamen aldehyde, Cyclosal, Lime aldehyde (Alpha-methyl-p-isopropyl phenyl propyl aldehyde), Methyl Nonyl Acetaldehyde, aldehyde C12 MNA (2-methyl-1-undecanal), Hydroxycitronellal, citronellal hydrate (7-hydroxy-3,7-dimethyl octan-1-al), Helional (alpha-methyl-3,4-(methylenedioxy)-hydrocinnamaldehyde, hydrocinnamaldehyde (3-phenylpropanal, 3-phenylpropionaldehyde), Intreleven aldehyde (undec-10-en-1-al), Ligustral, Trivertal (2,4-dimethyl-3-cyclohexene-1-carboxaldehyde), Jasmorange, satinaldehyde (2-methyl-3-tolylproionaldehyde, 4-dimethylbenzenepropanal), Lyral (4-(4-hydroxy-4-methyl pentyl)-3-cyclohexene-1-carboxaldehyde), Melonal (2,6-Dimethyl-5-Heptenal), Methoxy Melonal (6-methoxy-2,6-dimethylheptanal), methoxycinnamaldehyde (trans-4-methoxycinnamaldehyde), Myrac aldehyde isohexenyl cyclohexenyl-carboxaldehyde, trifernal ((3-methyl-4-phenyl propanal, 3-phenyl butanal), lilial, P.T. Bucinal, lysmeral, benzenepropanal (4-tert-butyl-alpha-methyl-hydrocinnamaldehyde), Dupical, tricyclodecylidenebutanal (4-Tricyclo5210-2,6decylidene-8butanal), Melafleur (1,2,3,4,5,6,7,8-octahydro-8,8-dimethyl-2-naphthaldehyde),

Methyl Octyl Acetaldehyde, aldehyde C-11 MOA (2-mehtyl deca-1-al), Onicidal (2,6,10-trimethyl-5,9-undecadien-1-al), Citronellyl oxyacetaldehyde, Muguet aldehyde 50 (3,7-dimethyl-6-octenyl) oxyacetaldehyde), phenylacetaldehyde, Mefranal (3-methyl-5-phenyl pentanal), Triplal, Vertocitral dimethyl tetrahydrobenzene aldehyde (2,4-dimethyl-3-cyclohexene-1-carboxaldehyde), 2-phenylproprionaldehyde, Hydrotropaldehyde, Canthoxal, anisylpropanal 4-methoxy-alpha-methyl benzenepropanal (2-anisylidene propanal), Cylcemone A (1,2,3,4,5,6,7,8-octahydro-8,8 -dimethyl-2-naphthaldehyde), and Precylcemone B (1-cyclohexene-1-carboxaldehyde).

Still other exemplary aldehydes include, but are not limited to, acetaldehyde (ethanal), pentanal, valeraldehyde, amylaldehyde, Scentenal (octahydro-5-methoxy-4,7-Methano-1H-indene-2-carboxaldehyde), propionaldehyde (propanal), Cyclocitral, beta-cyclocitral, (2,6,6-trimethyl-1-cyclohexene-1-acetaldehyde), Iso Cyclocitral (2,4,6-trimethyl-3-cyclohexene-1-carboxaldehyde), isobutyraldehyde, butyraldehyde, isovaleraldehyde (3-methyl butyraldehyde), methylbutyraldehyde (2-methyl butyraldehyde, 2-methyl butanal), Dihydrocitronellal (3,7-dimethyl octan-1-al), 2-Ethylbutyraldehyde, 3-Methyl-2-butenal, 2-Methylpentanal, 2-Methyl

Valeraldehyde, Hexenal (2-hexenal, trans-2-hexenal), Heptanal, Octanal, Nonanal, Decanal, Lauric aldehyde, Tridecanal, 2-Dodecanal, Methylthiobutanal, Glutaraldehyde, Pentanedial, Glutaric aldehyde, Heptenal, cis or trans-Heptenal, Undecenal (2-, 10-), 2,4-octadienal, Nonenal (2-, 6-), Decenal (2-, 4-), 2,4-hexadienal, 2,4-Decadienal, 2,6-Nonadienal, Octenal, 2,6-dimethyl 5-heptenal, 2-isopropyl-5-methyl-2-hexenal, Trifernal, beta methyl Benzenepropanal, 2,6,6-Trimethyl-1-cyclohexene-1-acetaldehyde, phenyl Butenal (2-phenyl 2-butenal), 2.Methyl-3(p-isopropylphenyl)-propionaldehyde, 3-(p-isopropylphenyl)-propionaldehyde, p-Tolylacetaldehyde (4-methylphenylacetaldehyde), Anisaldehyde (p-methoxybenzene aldehyde), Benzaldehyde, Vernaldehyde (1-Methyl-4-(4-methylpentyl)-3-cyclohexenecarbaldehyde), Heliotropin (piperonal) 3,4-Methylene dioxy benzaldehyde, alpha-Amylcinnamic aldehyde, 2-pentyl-3-phenylpropenoic aldehyde, Vanillin (4-methoxy 3-hydroxy benzaldehyde), Ethyl vanillin (3-ethoxy 4-hydroxybenzaldehyde), Hexyl Cinnamic aldehyde, Jasmonal H (alpha-n-hexyl-cinnamaldehyde), Floralozone, (para-ethyl-alpha,alpha-dimethyl Hydrocinnamaldehyde), Acalea (p-methyl-alpha-pentylcinnamaldehyde), methylcinnamaldehyde, alpha-Methylcinnamaldehyde (2-methyl 3-pheny propenal), alpha-hexylcinnamaldehyde (2-hexyl 3-phenyl propenal), Salicylaldehyde (2-hydroxy benzaldehyde), 4-ethyl benzaldehyde, Cuminaldehyde (4-isopropyl benzaldehyde), Ethoxybenzaldehyde, 2,4-dimethylbenzaldehyde, Veratraldehyde (3,4-dimethoxybenzaldehyde), Syringaldehyde (3,5-dimethoxy 4-hydroxybenzaldehyde), Catechaldehyde (3,4-dihydroxybenzaldehyde), Safranal (2,6,6-trimethyl-1,3-diene methanal), Myrtenal (pin-2-ene-1-carbaldehyde), Perillaldehyde L-4(1-methylethenyl)-1-cyclohexene-1-carboxaldehyde), 2,4-Dimethyl-3-cyclohexene carboxaldehyde, 2-Methyl-2-pentenal, 2-methylpentenal, pyruvaldehyde, formyl Tricyclodecan, Mandarin aldehyde, Cyclemax, Pino acetaldehyde, Corps Iris, Maceal, and Corps 4322.

In one embodiment, the malodor control composition includes a mixture of two or more volatile aldehydes selected from the group consisting of 2-ethoxy Benzylaldehyde, 2-isopropyl-5-methyl-2-hexenal, 5-methyl Furfural, 5-methyl-thiophene-carboxaldehyde, Adoxal, p-anisaldehyde, Benzylaldehyde, Bourgenal, Cinnamic aldehyde, Cymal, Decyl aldehyde, Floral super, Florhydral, Helional, Lauric aldehyde, Ligustral, Lyral, Melonal, o-anisaldehyde, Pino acetaldehyde, P.T. Bucinal, Thiophene carboxaldehyde, trans-4-Decenal, trans 2,4-Nonadienal, Undecyl aldehyde, and mixtures thereof.

In some embodiments, the malodor control composition includes fast reacting volatile aldehydes. “Fast reacting volatile aldehydes” refers to volatile aldehydes that either (1) reduce amine odors by 20% or more in less than 40 seconds; or (2) reduce thiol odors by 20% or more in less than 30 minutes.

Additionally, the individual volatile aldehydes or a various combination of the volatile aldehydes can be formulated into a malodor control composition. In certain embodiments, the volatile aldehydes may be present in an amount up to 100%, by weight of the malodor control composition, alternatively from 1% to about 100%, alternatively from about 2% to about 100%, alternatively from about 3% to about 100%, alternatively about 50% to about 100%, alternatively about 70% to about 100%, alternatively about 80% to about 100%, alternatively from about 1% to about 20%, alternatively from about 1% to about 10%, alternatively from about 1% to about 5%, alternatively from about 1% to about 3%, alternatively from about 2% to about 20%, alternatively from about 3% to about 20%, alternatively from about 4% to about 20%, alternatively from about 5% to about 20%, by weight of the composition.

In some embodiments where volatility is not important for neutralizing a malodor, the present invention may include poly-aldehydes, for example, di-, tri-, tetra-aldehydes. Such embodiments may include direct application to the compostable materials contained in one more containers of the composting appliance.

The malodor control composition of the present invention may include an effective amount of an acid catalyst to neutralize sulfur-based malodors. It has been found that certain mild acids have an impact on aldehyde reactivity with thiols in the liquid and vapor phase. It has been found that the reaction between thiol and aldehyde is a catalytic reaction that follows the mechanism of hemiacetal and acetal formation path. When the present malodor control composition contains an acid catalyst and contacts a sulfur-based malodor, the volatile aldehyde reacts with thiol. This reaction may form a thiol acetal compound, thus, neutralizing the sulfur-based odor. Without an acid catalyst, only hemi-thiol acetal is formed.

Suitable acid catalysts have a VP, as reported by Scifinder, in the range of about 0.001 ton to about 38 torr, measured at 25° C., alternatively about 0.001 torr to about 14 torr, alternatively from about 0.001 to about 1, alternatively from about 0.001 to about 0.020, alternatively about 0.005 to about 0.020, alternatively about 0.010 to about 0.020. The acid catalyst may be a weak acid. A weak acid is characterized by an acid dissociation constant, Kₐ, which is an equilibrium constant for the dissociation of a weak acid; the pKa being equal to minus the decimal logarithm of Kₐ. The acid catalyst may have a pKa from about 4.0 to about 6.0, alternatively from about 4.3 and 5.7, alternatively from about 4.5 to about 5, alternatively from about 4.7 to about 4.9. Suitable acid catalyst may include the following: Formic acid, acetic acid, trimethyl acetic acid, phenol (alkaline in liquid apps yet acidic in vapor phase), tiglic acid, caprylic acid, 5-methyl thiophene carboxylic acid, succinic acid, benzoic acid, mesitylenic acid.

Depending on the desired use of the malodor control composition, one may consider the scent character or the affect on the scent of the malodor control composition when selecting an acid catalyst. In some embodiments of the malodor control composition, it may be desirable to select an acid catalyst that provides a neutral to pleasant scent. Such acid catalysts may have a VP of about 0.001 torr to about 0.020 torr, measured at 25° C., alternatively about 0.005 torr to about 0.020 torr, alternatively about 0.010 torr to about 0.020 torr. Non-limiting examples of such acid catalyst include 5-methyl thiophene carboxaldehyde with carboxylic acid impurity, succinic acid, or benzoic acid.

The malodor control composition may include about 0.05% to about 5%, alternatively about 0.1% to about 1.0%, alternatively about 0.1% to about 0.5%, alternatively about 0.1% to about 0.4%, alternatively about 0.4% to about 1.5%, alternatively about 0.4% of an acid catalyst by weight of the malodor control composition.

The malodor control composition may have a pH from about 3 to about 8, alternatively from about 4 to about 7, alternatively from about, alternatively from about 4 to about 6.

The MCC of the present invention may comprise a malodor control polymer. On example of a malodor control polymer includes a polyamine polymer having a primary, secondary, and/or tertiary amine group. Another example is a hydrophobically modified malodor control polymer (HMP) that is formed from a polyamine polymer having a primary, secondary, and/or tertiary amine group that is modified with a hydrophobic group such as an alkyl, alkenyl, alkyloxide, or amide. Although the amine group has been modified, a HMP has at least one free and unmodified primary, secondary, and/or tertiary amine group, to react with malodorous components. Not wishing to be bound by theory, hydrophobic modification may increase a polymer's affinity for hydrophobic odors, thus enabling interactions between the odor molecules and active amine sites. In turn, HMPs may improve the breadth of malodor removal efficacy

HMPs for use herein may have a MW from about 150 to about 2*10⁶, alternatively from about 400 to about 10⁶, alternatively from about 5000 to about 10⁶.

Malodor control polymers suitable for use in the present invention are water-soluble or dispersible. In some embodiments, the primary, secondary, and/or tertiary amines of the polyamine chain are partially substituted rendering hydrophobicity while maintaining the desired water solubility. The minimum solubility index of a HMP may be about 2% (i.e. 2 g/100 ml of water). A suitable HMP for an aqueous MCC may have a water solubility percentage of greater than about 0.5% to 100%, alternatively greater than about 5%, alternatively greater than about 10%, alternatively greater than about 20%.

The MCC of the present invention may comprise a lower molecular weight polyol. Low molecular weight polyols with relatively high boiling points, as compared to water, such as ethylene glycol, diethylene glycol, triethylene glycol, propylene glycol, dipropylene glycol, and/or glycerin may be utilized as a malodor counteractant for improving odor neutralization of the MCC of the present invention. Some polyols, e.g., dipropylene glycol, are also useful to facilitate the solubilization of some perfume ingredients in the composition of the present invention.

The glycol used in the composition of the present invention may be glycerine, ethylene glycol, propylene glycol, dipropylene glycol, polyethylene glycol, propylene glycol methyl ether, propylene glycol phenyl ether, propylene glycol methyl ether acetate, propylene glycol n-butyl ether, dipropylene glycol n-butyl ether, dipropylene glycol n-propyl ether, ethylene glycole phenyl ether, diethylene glycol n-butyl ether, dipropylene glycol n-butyl ether, diethylene glycol mono butyl ether, dipropylene glycol methyl ether, tripropylene glycol methyl ether, tripropylene glycol n-butyl ether, other glycol ethers, or mixtures thereof. In one embodiment, the glycol used is ethylene glycol, propylene glycol, or mixtures thereof. In another embodiment, the glycol used is diethylene glycol.

Typically, the low molecular weight polyol is added to the composition of the present invention at a level of from about 0.01% to about 5%, by weight of the composition, alternatively from about 0.05% to about 1%, alternatively from about 0.1% to about 0.5%, by weight of the composition. The weight ratio of low molecular weight polyol to the HMP is from about 500:1 to about 4:1, alternatively from about 1:100 to about 25:1, alternatively from about 1:50 to about 4:1, alternatively about 4:1.

The MCC of the present invention may comprise a cyclodextrin. In some embodiments, the composition may include solubilized, water-soluble, uncomplexed cyclodextrin. As used herein, the term “cyclodextrin” includes any of the known cyclodextrins such as unsubstituted cyclodextrins containing from six to twelve glucose units, especially, alpha-cyclodextrin, beta-cyclodextrin, gamma-cyclodextrin and/or their derivatives and/or mixtures thereof.

Cyclodextrin molecules are described in U.S. Pat. No. 5,714,137, and U.S. Pat. No. 5,942,217. Suitable non-limiting levels of cyclodextrin are from about 0.1% to about 5% by weight of the MCC.

The MCC of the present invention may comprise a buffering agent. The composition of the present invention may include a buffering agent which may be a dibasic acid, carboxylic acid, or a dicarboxylic acid like maleic acid. The acid may be sterically stable, and used in this composition solely for maintaining the desired pH. The composition may have a pH from about 6 to about 8, alternatively from about 6 to about 7, alternatively about 7, alternatively about 6.5.

In some embodiments, when the HMP is not water soluble, it may be desirable to adjust pH of the MCC from about 6 to about 8, alternatively from about 6 to about 7, alternatively about 7, alternatively about 6.5.

Carboxylic acids such as citric acid may act as metal ion chelants and can form metallic salts with low water solubility. As such, in some embodiments, the MCC is essentially free of citric acids. The buffer can be alkaline, acidic or neutral.

Other suitable buffering agents for MCC of this invention include biological buffering agents. Some examples are nitrogen-containing materials, sulfonic acid buffers like 3-(N-morpholino)propanesulfonic acid (MOPS) or N-(2-Acetamido)-2-aminoethanesulfonic acid (ACES), which have a near neutral 6.2 to 7.5 pKa and provide adequate buffering capacity at a neutral pH. Other examples are amino acids such as lysine or lower alcohol amines like mono-, di-, and tri-ethanolamine. Other nitrogen-containing buffering agents are tri(hydroxymethyl)amino methane (HOCH2)3CNH3 (TRIS), 2-amino-2-ethyl-1,3-propanediol, 2-amino-2-methyl-propanol, 2-amino-2-methyl-1,3-propanol, disodium glutamate, N-methyl diethanolamide, 2-dimethylamino-2-methylpropanol (DMAMP), 1,3-bis(methylamine)-cyclohexane, 1,3-diamino-propanol N,N′-tetra-methyl-1,3-diamino-2-propanol, N,N-bis(2-hydroxyethyl)glycine (bicine) and N-tris (hydroxymethyl)methyl glycine (tricine). Mixtures of any of the above are also acceptable.

The compositions may contain at least about 0%, alternatively at least about 0.001%, alternatively at least about 0.01%, by weight of the composition, of a buffering agent. The composition may also contain no more than about 1%, alternatively no more than about 0.75%, alternatively no more than about 0.5%, by weight of the composition, of a buffering agent.

The MCC of the present invention may comprise a solubilizer. The composition of the present invention may contain a solubilizing aid to solubilize any excess hydrophobic organic materials, particularly any perfume materials, and also optional ingredients (e.g., insect repelling agent, antioxidant, etc.) which can be added to the composition, that are not readily soluble in the composition, to form a clear solution. A suitable solubilizing aid is a surfactant, such as a no-foaming or low-foaming surfactant. Suitable surfactants are nonionic surfactants, cationic surfactants, amphoteric surfactants, zwitterionic surfactants, and mixtures thereof.

In some embodiments, the MCC contains nonionic surfactants, cationic surfactants, and mixtures thereof. In one embodiment, the MCC contains hydrogenated castor oil. One suitable hydrogenated castor oil that may be used in the present composition is Basophor™, available from BASF.

When the solubilizing agent is present, it is typically present at a level of from about 0.01% to about 3%, alternatively from about 0.05% to about 1%, alternatively from about 0.01% to about 0.05%, by weight of the MCC.

The MCC of the present invention may comprise an antimicrobial compound. The composition of the present invention may include an effective amount of a compound for reducing microbes in the air. Antimicrobial compounds are effective on gram negative and gram positive bacteria and fungi. Microbial species include Klebsiella pneumoniae, Staphylococcus aureus, Aspergillus niger, Klebsiella pneumoniae, Steptococcus pyogenes, Salmonella choleraesuis, Escherichia coli, Trichophyton mentagrophytes, and Pseudomonas aeruginosa. In some embodiments, the antimicrobial compounds are also effective on viruses such H1-N1, Rhinovirus, Respiratory Syncytial, Poliovirus Type 1, Rotavirus, Influenza A, Herpes simplex types 1 & 2, Hepatitis A, and Human Coronavirus.

Water-soluble antimicrobial compounds include organic sulfur compounds, halogenated compounds, cyclic organic nitrogen compounds, low molecular weight aldehydes, quaternary compounds, dehydroacetic acid, phenyl and phenoxy compounds, or mixtures thereof. The antimicrobial compound may be present in an amount from about 500 ppm to about 7000 ppm, alternatively about 1000 ppm to about 5000 ppm, alternatively about 1000 ppm to about 3000 ppm, alternatively about 1400 ppm to about 2500 ppm, by weight of the composition.

The MCC of the present invention may comprise an aqueous carrier. If small amounts of low molecular weight monohydric alcohols are present in the composition of the present invention due to the addition of these alcohols to such things as perfumes and as stabilizers for some preservatives, the level of monohydric alcohol may be less than about 6%, alternatively less than about 3%, alternatively less than about 1%, by weight of the composition.

The MCC of the present invention may include a preservative. The MCC of the present invention may include one or more ingredients described in the U.S. Pat. Publications of the respective U.S. patent application Ser. Nos. 12/962691; 13/081559; 12/904261; 13/006644; 13/249616; and 61/622030 (incorporated herein by reference). The ingredients described in these publication may be in addition or in lieu of the aforementioned ingredients.

In one embodiment, an odor sensor is employed. One or more odor sensors may be employed to assess the malodor: contained in the container and/or air space contained in the container; and/or being emitted from the contents contained in the container (of the composting appliance). An example of an odor sensor may include one described by U.S. Pat. No. 6,093,308. The odor sensor can be electrically coupled to a MCC dispenser (wherein the MCC dispenser is capable of dispensing MCC in or adjacent the home composting appliance).

The present invention is directed to the use of MCC in composting, preferably indoor composting, alternatively in or adjacent a composting appliance. A first aspect of the invention provides a composting appliance (preferably for in-home use), in the simplest sense, comprises a reacting container or designated area suitable for containing food scraps and other suitable materials for composting. The reacting container is typically from about 1 liter to about 100 liters, alternatively from 25 liters to 75 liters, alternatively from 35 liters to 65 liters, alternatively combinations thereof. The appliance may contain a lid or other similar means of enclosing/accessing the reacting container (and the contents contained therein). The lid mitigates the emission of malodor emitting from materials contained in the reacting container. The home composting appliance typically comprising a mixing means that will mix the contents of the reacting container to facilitate the composting processes (e.g., improve air flow). The mixing means may be engaged on a periodic basis or a continuous basis or a combination thereof. The appliance may optionally contain a heater means (e.g., electrical heating jacket) to heat the reacting container and/or the contents contained in the container to facilitate the composting processes since many composting microbes prefer temperatures above those of ambient (i.e., above about 21° C.). Preferably the reacting container is insulated so that heating is more efficient/economical. The lid may serve the function of preventing the contents of the container from being splattered out during the mixing process and/or keeping heat contained in the container/contents (should a heater be used). In some composting appliances, there is a curing container. In other words, some appliances have a two stage process—an initial active phase and a subsequent curing phase. The curing phase is typically longer in time than the active phase, but preferably about the same time. The curing container may have the volumetric parameters as previously described for the reacting container. Non-limiting examples of composting appliances include those described in JP 3601973 B2; and US 2008/0209967 A1. In one embodiment, the MCC of the present invention are administered to a composting appliance, alternatively specifically to an active container of an in-home composting appliance. In another embodiment, the MCC are administered to a curing container of an in-home composting appliance. In yet other embodiments, the MCC are administered to both the active container and the curing container of the in-home composting appliance. In one embodiment, in-home composting is conducted in a bin, bucket or bag, alternatively without mechanical mixing and/or without external heating. In yet another embodiment, the contents of the appliance either pre- or post-composting may be a contained in a bag, preferably a biodegradable bag. The volume of the bag may be similar to the volume previously expressed for the reacting container.

One aspect of the invention provides for a composting appliance comprising a MCC dispenser that is in fluid communication with the reacting container and/or curing container (or additional containers the appliance may have). The dispenser further comprises a dispensing container that is capable of containing one or more MCC of the present invention, and preferably dispensing portions of the MCC to the container or air space contained in the container - of the composting appliance. In one embodiment, the volume of the MCC dispensing container is from about 10 ml to about 4,000 ml (or more), alternatively about 100 ml to about 2,000 ml, alternatively from about 500 ml to about 1,000 ml, alternatively combinations thereof. The dispensing of the MCC by the dispenser, in one embodiment, is actuated by one or more events. The event may be the user opening the lid or pressing a button on the appliance or a pre-determined time interval (e.g., daily) or a sensor detecting a stimulus (e.g., malodor, the weight of pre-compost being added to the reacting container, etc).

The appliance may contain more than one dispenser. There may be a first dispenser dedicated to the reacting container, and a second dispenser dedicated to the curing container. Alternatively, there may be a first dispenser dedicated to dispensing a first MCC to the reacting container and a second dispenser dedicated to dispensing a second MCC also to the reacting container. A third and fourth dispenser may be dedicated to dispensing a respective third and fourth MCC to the curing container. Combinations of these dispensers are also contemplated.

In one embodiment, the compostable material comprises food scraps (e.g., spoiled or uneaten food). In another embodiment, compostable material is free or substantially free of recyclable materials (such as glass and aluminum). The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A composting appliance comprising:
(a) at least one container capable of containing compostable materials;
(b) a heater configured to heat the container or the compostable materials contained in the container;
(c) an optional mixing element configured to mix the compostable materials contained in the container;
(d) an optional vent in fluid communication between the compostable materials contained in the container and the atmospheric air outside of the composting appliance.
(e) a passive or energized malodor control composition dispensing device configured to dispense a malodor control composition.

2. The appliance of claim 1, further comprising a vial containing the malodor control composition, wherein the vial is functionally attached to the dispensing device.

3. The appliance of claim 2, wherein the vial is functionally releasably attached to the dispensing device.

4. The appliance of claim 3, wherein the dispensing device is active and comprises a wick and heater or a vibrator

5. The appliance of claim 2, where the appliance comprises the vent and wherein in the dispensing device is an air filter and functionally attached to the vent.

6. The appliance of claim 5, wherein the air filter comprises activated carbon.

7. The appliance of claim 6, further comprises a fan configured in the vent and capable of exhausting gaseous emission emitting from the container through the vent.

8. The appliance of claim 2, wherein the dispensing device is configured to dispense the malodor control composition into the container.

9. The appliance of claim 8, wherein the dispensing device is configured to meter pre-determined volumes of the malodor eliminating composition.

10. The appliance of claim 1, wherein the malodor control composition comprises from about 50% to about 100% by weight of the composition of at least one perfume ingredient that has a Kovat's Index from about 600 to about 1800.

11. The appliance of claim 10, wherein the composition comprises at least five perfume ingredients having a Kovat's Index from about 600 to about 1800.

12. The appliance of claim 1, wherein the malodor control composition comprises perfume ingredients, and wherein the perfume ingredients are selected from:
(i) a first group of ingredients having a boiling point of about 20° C. to about 250° C. and a ClogP value from about −2 to about 3;
(ii) a second group of ingredients having a boiling point of about 20° C. to about 250° C. and a ClogP value from about 3 to about 9;
(iii) a third group of ingredients having a boiling point of about 250° C. to about 400° C. and a ClogP value from about −2 to about 3; a fourth group of ingredients having a boiling point of about 250° C. to about 400° C. and a ClogP of about 3 to about 9; or
(iv) a combination thereof.

13. The appliance of claim 10, wherein the malodor control composition comprises perfume ingredients, and wherein the perfume ingredients are selected from:
(i) a first group of ingredients having a boiling point of about 20° C. to about 250° C. and a ClogP value from about −2 to about 3;
(ii) a second group of ingredients having a boiling point of about 20° C. to about 250° C. and a ClogP value from about 3 to about 9;
(iii) a third group of ingredients having a boiling point of about 250° C. to about 400° C. and a ClogP value from about −2 to about 3; a fourth group of ingredients having a boiling point of about 250° C. to about 400° C. and a ClogP of about 3 to about 9; or
(iv) a combination thereof.

14. The appliance of claim 11, wherein the malodor control composition comprises perfume ingredients, and wherein the perfume ingredients are selected from:
(i) a first group of ingredients having a boiling point of about 20° C. to about 250° C. and a ClogP value from about −2 to about 3;
(ii) a second group of ingredients having a boiling point of about 20° C. to about 250° C. and a ClogP value from about 3 to about 9;
(iii) a third group of ingredients having a boiling point of about 250° C. to about 400° C. and a ClogP value from about −2 to about 3; a fourth group of ingredients having a boiling point of about 250° C. to about 400° C. and a ClogP of about 3 to about 9; or
(iv) a combination thereof.

15. A method of eliminating odor comprising the steps:
a) providing a container configured to receive compostable materials;
b) containing compostable materials in the container;
c) composting the compostable materials contained in the container
d) emitting malodor from the container containing the compostable materials;
e) dispensing a malodor control composting in fluid communication the compostable materials contained in the container to eliminate the emitted malodor.

16. The method of claim 15, wherein the malodor control composition comprises from about 50% to about 100% by weight of the composition of at least one perfume ingredient that has a Kovat's Index from about 600 to about 1800.

17. The method of claim 16, wherein the malodor control composition comprises perfume ingredients, and wherein the perfume ingredients are selected from:
(i) a first group of ingredients having a boiling point of about 20° C. to about 250° C. and a ClogP value from about −2 to about 3;
(ii) a second group of ingredients having a boiling point of about 20° C. to about 250° C. and a ClogP value from about 3 to about 9;
(iii) a third group of ingredients having a boiling point of about 250° C. to about 400° C. and a ClogP value from about −2 to about 3; a fourth group of ingredients having a boiling point of about 250° C. to about 400° C. and a ClogP of about 3 to about 9; or
(iv) a combination thereof.

18. A method of eliminating odor in a home composting appliance comprising the steps:
(a) providing a unit dose article comprising a malodor control composition;
(d) administering the article into a container of a home composting appliance wherein the container is capable of containing compostable materials.

19. The method of claim 18, wherein the malodor control composition comprises from about 50% to about 100% by weight of the composition of at least one perfume ingredient that has a Kovat's Index from about 600 to about 1800.

20. The method of claim 19, wherein the malodor control composition comprises perfume ingredients, and wherein the perfume ingredients are selected from:
(i) a first group of ingredients having a boiling point of about 20° C. to about 250° C. and a ClogP value from about −2 to about 3;
(ii) a second group of ingredients having a boiling point of about 20° C. to about 250° C. and a ClogP value from about 3 to about 9;
(iii) a third group of ingredients having a boiling point of about 250° C. to about 400° C. and a ClogP value from about −2 to about 3; a fourth group of ingredients having a boiling point of about 250° C. to about 400° C. and a ClogP of about 3 to about 9; or
(iv) a combination thereof.
